(19) United States
(12) Patent Application Publication
(10) Pub. No.: US 2025/0276126 A1
(43) Pub. Date: Sep. 4, 2025

(54) INFUSION SET AUXILIARY DEVICE AND AN INFUSION SET SYSTEM FOR VASCULAR INTERVENTION

(71) Applicant: Shenzhen Nanshan People's Hospital, Shenzhen (CN)

(72) Inventor: Chuanming WANG, Shenzhen (CN)

(73) Assignee: Shenzhen Nanshan People's Hospital, Shenzhen (CN)

(21) Appl. No.: 19/188,806

(22) Filed: Apr. 24, 2025

Related U.S. Application Data
(63) Continuation of application No. PCT/CN2023/126228, filed on Oct. 24, 2023.

(30) Foreign Application Priority Data
Oct. 27, 2022 (CN) 202222850617.5

(51) Int. Cl. A61M 5/172

(52) U.S. Cl. CPC A61M 5/172

(57) ABSTRACT

An infusion set auxiliary device and an infusion set system for vascular intervention are provided. A fixing seat is convex on a bottom plate and has a top surface, a bottom surface and a side surface which is opposite to the bottom plate. The fixing seat includes a plurality of fixing holes arranged along a first direction, and each fixing hole is used for holding a drip pot of one infusion set. The fixing hole penetrates through the side surface, the top surface and the bottom surface to form a first opening, a second opening and a third opening respectively, the sizes of the first opening, the second opening and the third opening satisfy corresponding relationships with the outside diameter of the infusion tube and the outside diameter of the drip pot. The application can arrange multiple infusion sets neatly to facilitate differentiation.

### CROSS-REFERENCE TO RELATED APPLICATION(S)

This application is a continuation of International Application No. PCT/CN2023/126228, filed Oct. 24, 2023, which claims priority to Chinese Patent Application No. 202222850617.5, filed Oct. 27, 2022, the entire disclosures of which are incorporated herein by reference.

### TECHNICAL FIELD

The present disclosure relates to the technical field of vascular intervention medical equipment, and in particular to an infusion set auxiliary device and an infusion set system for vascular intervention used in nerve intervention, cardiovascular intervention, peripheral intervention and other operations.

### BACKGROUND

In a course of vascular interventional therapy, such as nerve intervention, cardiovascular intervention, and peripheral intervention, it may be necessary to use multiple infusion sets to flush the interventional catheter at the same time to prevent intraductal thrombosis or distal vascular embolization events. For example, in neurovascular interventional embolization of cerebral aneurysms, a femoral artery sheath, a guide catheter, an intermediate catheter, a stent micro catheter, and a spring coil embolization catheter these five catheters all need to be flushed with heparin saline. For most vascular interventions, 3-5 infusion sets are required to flush the catheter. Usually, the infusion set includes a bottle stopper puncture set, an infusion tube, a drip pot, a drip rate controller, etc. When the number of the infusion sets is too much, and the infusion tube usually is suspended, resulting in the operation table is mussy, it is not suitable to distinguish different infusion sets, and it is not suitable to observe the dripping situation of the drip pots. It is difficult for the operator or assistant to adjust the drip rate for the infusion set that needs to be adjusted, replace the joint, and close the pipe, which brings obstacles to the risk control of surgical operation.

In addition, due to the number of infusion sets used for flushing catheter is much, and infusion bottles are hung on the infusion rack in a circular distribution, resulting in some of the drip pots being blocked by other infusion tubes and other drip pots, and it is not easy to observe the liquid dripping rate of all drip pots. The drip rate of each infusion set for flushing is manually controlled and the drip rate is subjectively estimated. If the drip rate controller is slipped, the drip rate may be too fast and the fluid entering the catheter may be too much, thus increasing the risk of pulmonary edema, and if the drip rate is slowed or the drip is stopped, intraductal thrombosis may occur, thus increasing the risk of vascular embolism.

Present application aims to develop an infusion set auxiliary device for vascular intervention and an infusion set system, which provides an infusion set arrangement system for flushing with advantages such as clear marking, orderly arrangement, intuitive observation, and provides an automatic electronic display, adjustment of drip rate and fault alarm system, which can provide flushing convenience for vascular intervention operation, and improve the safety and effectiveness of interventional surgery.

### SUMMARY

present application provides an infusion set auxiliary device and infusion set system for vascular intervention, which is used to organize infusion sets for executing vascular interventional flushing operation.

In a first aspect, the present application provides an infusion set auxiliary device for vascular intervention, the infusion set auxiliary device includes:
a bottom plate;
a fixing seat, the fixing seat is arranged on the bottom plate, the fixing seat has a top surface, a bottom surface opposite to the top surface, and a side surface connected between the top surface and the bottom surface, the side surface is arranged opposite to the bottom plate, the fixing seat is provided with a plurality of fixing holes arranged in a first direction sequentially, each fixing hole is at least used to contain a drip pot of a disposable medical infusion set;
therein, each fixing hole penetrates through the side surface and forms a first opening on the side surface, and a size of the first opening along the first direction is greater than or equal to the outside diameter of the infusion tube of the infusion set and less than the outside diameter of the drip pot;
therein, each fixing hole penetrates through the top surface and forms a second opening on the top surface, the second opening is connected with the first opening, and a size of the second opening is greater than or equal to the outside diameter of the drip pot;
therein, the fixing hole penetrates through the bottom surface and forms a third opening on the bottom surface, the third opening is connected with the first opening, and a size of the third opening is greater than or equal to the outside diameter of the infusion tube and less than the outside diameter of the drip pot.

The infusion set auxiliary device provided in present application, through providing a plurality of fixing holes arranged on the fixed base sequentially, and the size of the first opening of each fixing hole is greater than or equal to the outside diameter of the infusion tube and less than the outside diameter of the drip pot, so as to facilitate the infusion tube connected the bottom of the drip pot enters the fixing hole through the first opening, and the size of the second opening of the fixing hole is greater than or equal to the outside diameter of the drip pot, so that the drip pot can be placed in the fixing hole from an upper side of the fixing hole, and the size of the third opening of the fixing hole is greater than or equal to the outside diameter of the infusion tube of the infusion set and smaller than the outside diameter of the drip pot, thus to hold up the drip pot. According to that, the fixing hole can contain the drip pot of the infusion set, and due to the fixing holes are arranged sequentially, the drip pots of multiple infusion sets can be arranged in order, then, the drip pots and infusion tubes of multiple infusion sets are sorted out neatly, which effectively solves the problem that the operation table is mussy, and it is not suitable to distinguish different infusion sets, and it is difficult for the operator or assistant to adjust the drip rate, replace the joint, and close the pipe caused by the number of infusion sets are too much.

In a possible embodiment, the third opening comprises a support opening and a guide channel, the support opening is concentrically arranged with the second opening, the guide channel is connected between the support opening and the first opening, and a size of the support opening is greater than or equal to the outside diameter of the infusion tube and less than the outside diameter of the drip pot. A size of the guide channel along the first direction is greater than or equal to the outside diameter of the infusion tube and less than the size of the support opening.

In a possible embodiment, the guide channel is deviated from the geometric center of the support opening, and the guide channel is deviated to a right circumferential tangent of the support opening to avoid the infusion tube being pulled directly from the guide channel.

In a possible embodiment, the bottom plate is provided with a plurality of light-transmitting holes arranged sequentially along the first direction, each light-transmitting hole is connected with one corresponding fixing hole;
the infusion set auxiliary device further includes a plurality of light-emitting elements arranged on a side of the bottom plate deviating from the fixing seat, and each light-emitting element emits light toward to the corresponding fixing hole through the corresponding light-transmitting hole.

In a possible embodiment, the infusion set auxiliary device further includes a plurality of light-transmitting glasses, each light-transmitting glass is arranged in one corresponding light-transmitting hole.

In a possible embodiment, the light-transmitting glass comprises a colorless light-transmitting glass and a colored light-transmitting glass spliced together, the colored light-transmitting glass is adjacent to the third opening relative to the colorless light-transmitting glass, and the colored light-transmitting glass is used for setting at a position at least relative to liquid at the bottom of the drip pot.

In a possible embodiment, the colors of the colored light-transmitting glasses in at least two of the light-transmitting holes are different, and the colors of the colored light-transmitting glasses may comprise: red, green, blue, purple, orange.

In a possible embodiment, the infusion set auxiliary device further includes a drip controller, the drip controller is located outside the infusion tube at the top of the drip pot to electronically control the drip rate of the drip in the drip pot instead of a traditional manual control device for the drip rate of the infusion set.

In a possible embodiment, the infusion set auxiliary device further includes at least one drip detector each arranged on an inner wall of the corresponding fixing hole, and the drip detector is set relative to the drip of the drip pot to detect the drip rate of the drip in the drip pot.

In a possible embodiment, the infusion set auxiliary device further includes at least one liquid detector each sets with respect to an infusion tube connected to the top of the drip pot, and the liquid detector is used for detecting whether the infusion tube at the top of the drip pot has liquid;
the infusion set auxiliary device also comprises an alarm, the alarm is electrically connected with the drip detector and the liquid detector, the alarm is used to generate an alarm signal when the drip rate detected by the drip detector is 0 and the liquid in the infusion tube detected by the liquid detector is empty;
the infusion set auxiliary device further comprises a display device that electrically connects the drip detectors and the liquid detectors, and the display device is used to display the drip rate in the drip pot and display information about whether there is liquid in the infusion tube connected to the top of the drip pot.

In a second aspect, the application further provides an infusion set system for vascular intervention, the infusion set system includes a plurality of infusion sets and the above infusion set auxiliary device, each infusion set includes a drip pot arranged in the fixing hole of the infusion set auxiliary device.

### BRIEF DESCRIPTION OF THE DRAWINGS

In order to more clearly illustrate the technical scheme of embodiments of the present application, brief introduction of the drawings required to be used in the embodiments are presented below.

FIG. 1 is a structural diagram of an infusion set auxiliary device for vascular intervention provided in present application;

FIG. 2 is a structural diagram of an infusion set system for vascular intervention provided in present application;

FIG. 3 is a top view of the infusion set auxiliary device provided in FIG. 1;

FIG. 4 is an upward view of the infusion set auxiliary device provided in FIG. 1;

FIG. 5 is a section view of the infusion set auxiliary device along a X-Z plane provided in FIG. 1;

FIG. 6 is a rear view of the infusion set auxiliary device provided in FIG. 1;

FIG. 7 is a circuit structure block diagram of the infusion set auxiliary device provided in FIG. 1.

### ILLUSTRATION FOR LABELS OF THE DRAWINGS

infusion set auxiliary device—100; Bottom plate—10; Fixing seat—20; Top surface—201; Bottom surface—202; Side surface—203; fixing hole—21; Infusion set—200; Drip pot—210; First opening—211; Infusion tube—220; Second opening—212; Third opening—213; Support opening—213a; Guide channel—213b; Light—transmitting hole—101; Light source box—30; Light—emitting element—31; Light—transmitting glass—40; Colorless light—transmitting glass—41; Colored light—transmitting glass—42; Drip detector—50; Liquid detector—60; Alarm—70; Display device—80; Processor—90.

### DETAILED DESCRIPTION

The technical scheme in the embodiments of present application will be clearly and completely described in combination with the drawings attached to the embodiments of present application. Obviously, the embodiments described are only part of the embodiments of present application, but not all embodiments. In addition, the phrase “embodiment” in present application means that a particular feature, structure or characteristic described in conjunction with an embodiment may be included in at least one embodiment of present application. The presence of the phrase “embodiment” at various positions in the specification does not refer to the same embodiment, and is also not independent embodiment or alternative embodiment that is mutually exclusive with other embodiments. It is understood, both explicitly and implicitly, by those skilled in the art that, the embodiments described in the present application may be combined with other embodiments.

In some vascular interventional therapy scenarios, multiple infusion sets are used simultaneously to flush the interventional catheter, for example, in neurovascular interventional embolization of cerebral aneurysms, a femoral artery sheath, a guide catheter, an intermediate catheter, a stent micro catheter, and a spring coil embolization catheter these five catheters all need to be flushed with heparin saline. For most vascular interventions, 3-5 infusion sets are required to flush the catheter. Usually, the infusion set includes a bottle stopper puncture set, an infusion tube, a drip pot, a drip rate controller, etc. When the number of the infusion sets is too much, and the infusion tube usually is suspended, resulting in the operation table is mussy, it is not suitable to distinguish different infusion sets, and it is not suitable to observe the dripping situation of the drip pots. It is difficult for the operator or assistant to adjust the drip rate for the infusion set that needs to be adjusted, replace the joint, and close the pipe, which brings obstacles to the risk control of surgical operation. Present application aims to develop an infusion set auxiliary device for vascular intervention and an infusion set system, which provides an infusion set arrangement system for flushing with advantages such as clear marking, orderly arrangement, intuitive observation, and provides an automatic electronic adjustment of drip rate and fault alarm system, which can provide flushing convenience for vascular intervention operation, and improve the safety and effectiveness of interventional surgery.

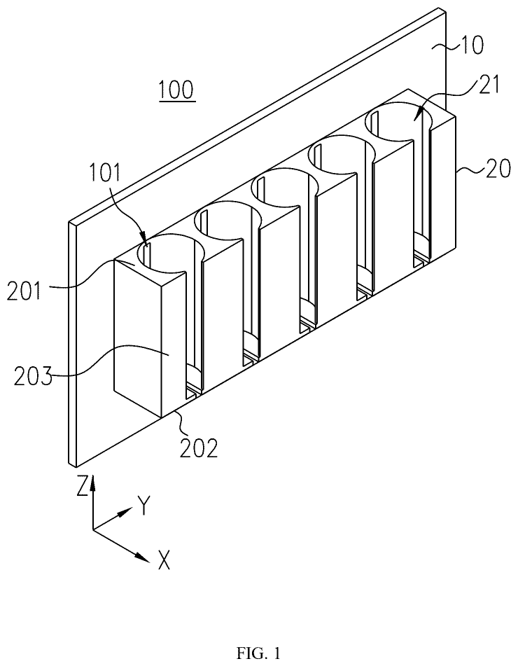

Please refer to FIG. 1, present application provides an infusion set auxiliary device 100 for vascular intervention, the infusion set auxiliary device 100 includes a bottom plate 10 and a fixing seat 20.

The bottom plate 10 may be plate-like, and its shape is not specified. Alternately, the shape of the bottom plate 10 is rectangular. The volume of the bottom plate 10 is not specified in present application. Alternately, the bottom plate 10 is placed upright as shown in FIG. 1, the volume of the bottom plate 10 is relatively small, e.g. 195 mm (millimeter) width*110 mm height*4 mm thickness. The infusion set auxiliary device 100 also includes a cross bar with a length of 200 mm. A cross bar connection point is arranged on a rear surface (the surface away from the fixing seat 20) of the bottom plate 10 to facilitate the subsequent installation of a light box (the light box is described in the following section), and also facilitate to fix an interventional surgical rod in an neurovascular interventional therapy.

The fixing seat 20 is convexly arranged on the bottom plate 10. The bottom plate 10 and the fixing seat 20 can be formed integratedly. The shape of the fixing seat 20 is not specified. Alternately, the fixed seat 20 is rectangular.

In order to facilitate description, in FIG. 1, a X-axis direction is defined as the width direction (forward or backward direction) of the fixing seat 20, a Y-axis direction is defined as the length direction (left-right direction) of fixing seat 20, and a Z-axis direction is defined as the height direction (up-down direction) of the fixing seat 20.

The fixing seat 20 has a top surface 201, a bottom surface 202 opposite to the top surface 201, and a side surface 203 connected between the top surface 201 and the bottom surface 202. The side surface 203 is arranged opposite to the bottom plate 10. The top surface 201 faces the top side/upper side, that is, the positive direction of the Z axis, and the bottom surface 202 faces the bottom side, that is, the negative direction of the Z axis. The side surface 203 faces the front side, that is, the positive direction of the X-axis.

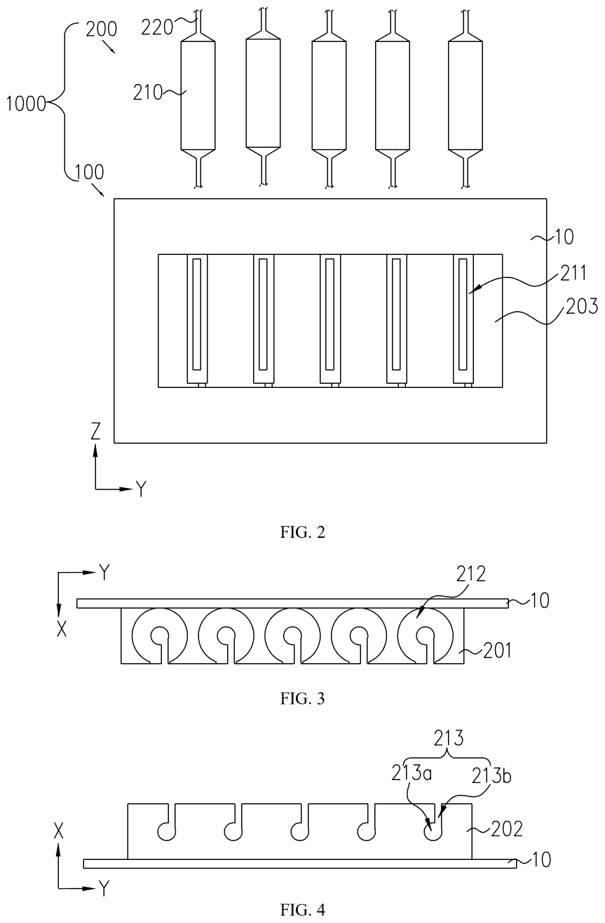

Please referring to FIG. 1 and FIG. 2, the fixing seat 20 is provided with a plurality of fixing holes 21 arranged along a first direction sequentially. The first direction is the Y-axis direction. Each fixing hole 21 is at least used for containing a drip pot 210 of a disposable medical infusion set 200. The present application does not make a specific limit on the number of fixing holes 21, for example, the number of fixing holes 21 may be 2, 3, 4, 5, 6 or more than 6, etc.

Each fixing hole 21 is set along the Z-axis direction, namely is extended along the Z-axis direction, so that the drip pot 210 is arranged along the vertical direction. Alternately, the fixing hole 21 can roughly be a cylindrical hole.

Referring to FIG. 1 and FIG. 2, the fixing hole 21 penetrates through the side surface 203 and forms a first opening 211 on the side surface 203, and a size (width) of the first opening 211 along the first direction is greater than or equal to the outside diameter of an infusion pipe 220 and is smaller than the outside diameter of the drip pot 210, so that the infusion pipe 220 connected to the bottom of the drip pot 210 is facilitated to enter the fixing hole 21 through the first opening 211.

Referring to FIG. 1 and FIG. 3, the fixing hole 21 penetrates through the top surface 201 and forms a second opening 212 on the top surface 201, the second opening 212 is connected to the first opening 211, and a size of the second opening 212 is greater than or equal to the outside diameter of the drip pot 210, so that the drip pot 210 can be placed in the fixing hole 21 from the upper side of the fixing hole 21.

Referring to FIG. 1 and FIG. 4, the fixing hole 21 penetrates through the bottom surface 202 and forms a third opening 213 on the bottom surface 202, the third opening 213 is connected with the first opening 211, a size of the third opening 213 is greater than or equal to the outside diameter of the infusion pipe 220 of the infusion apparatus 200 and is smaller than the outside diameter of the drip pot 210, so that the infusion pipe 220 of the infusion apparatus 200 can pass through the third opening 213, and the bottom wall of the fixing hole 21 can hold up the drip pot 210, and the drip pot 210 thus is contained in the fixing hole 21.

When placing the infusion set 200 on the infusion set auxiliary device 100, first, the infusion tube 220 at the bottom of the drip pot 210 enters the fixing hole 21 through the first opening 211, and then the drip pot 210 enters the fixing hole 21 from the upper side of the fixing hole 21 until the bottom of the drip pot 210 is connected to the bottom wall of the fixing hole 21, so that the drip pot 210 is arranged in the fixing hole 21 of the fixing seat 20. The drip pots 210 of multiple infusion sets 200 are arranged in different fixing holes 21 in the above way, so as to arrange the infusion tubes 220 and drip pots 210 of different infusion set 200, thus facilitate to quickly adjust the drip rate, replace the joint, and close the pipe for the infusion set 200 that is needed. It also avoids some of drip pots 210 are blocked by other infusion tubes 220 and other drip pots 210, thus facilitate to observe the liquid dripping rate of all drip pots.

The fixing seat 20 provided in this embodiment is provided with 5 fixing holes 21b arranged neatly, which can organize 1-5 infusion sets 200 (one to five infusion sets 200) and facilitate the arrangement and use of the 1-5 infusion sets 200.

The first opening 211 can be used as an observation window to observe the liquid in the drip pot 210 and the drip rate of the drip pot 210. In present application, the observation window is a long rectangle set along the Z axis. The present application does not specify the width of the observation window along the Y-axis direction, such as, the width of the observation window can be 10 mm. The width of the first opening 211 is less than the outer diameter of the drip pot 210 to avoid the drip pot 210 contained in the fixing hole 21 being removed from the first opening 211.

The size of the second opening 212 are not specified in present application. Alternately, the size of the second opening 212 may be larger than the outside diameter of drip pots 210 of all sizes, thus, any drip pot 210 can be inserted into the fixing hole 21 from above, and the infusion set auxiliary device 100 provided in present application can be used with all types of infusion sets 200.

Alternately, the size of the second opening 212 is 25 mm (but not limited to this size), which can be compatible with the infusion sets 200 commonly used from different manufacturers, thus the second opening 212 has strong compatibility.

The infusion set auxiliary device 100 provided in present application, through providing a plurality of fixing holes 21 arranged on the fixed base 20 sequentially, and the size of the first opening 211 of each fixing hole 21 is greater than or equal to the outside diameter of the infusion tube 220 and less than the outside diameter of the drip pot 210, so as to facilitate the infusion tube 220 connected the bottom of the drip pot 210 enters the fixing hole 21 through the first opening 211, and the size of the second opening 212 of the fixing hole 21 is greater than or equal to the outside diameter of the drip pot 210, so that the drip pot 210 can be placed in the fixing hole 21 from an upper side of the fixing hole 21, and the size of the third opening 213 of the fixing hole is greater than or equal to the outside diameter of the infusion tube 220 of the infusion set 200 and smaller than the outside diameter of the drip pot 210, thus to hold up the drip pot 210. According to that, the fixing hole 21 can contain the drip pot 210 of the infusion set 200, and due to the fixing holes 21 are arranged sequentially, the drip pots 210 of multiple infusion sets 200 can be arranged in order, then, the drip pots 210 and infusion tubes 220 of multiple infusion sets 200 are sorted out neatly, which effectively solves the problem that the operation table is mussy, and it is not suitable to distinguish different infusion sets 200, and it is difficult for the operator or assistant to adjust the drip rate, replace the joint, and close the pipe, caused by the number of infusion sets 200 are too much.

Alternately, please referring to FIG. 4, the third opening 213 includes a support opening 213a and a guide channel 213b. The support opening 213a is concentrically arranged with the second opening 212. Optionally, the support opening 213a and the second opening 212 may both be circular. A center of the support opening 213a is collinear with a center of the second opening 212 and the collinear is arranged/extended along the Z-axis direction, namely a connection line between the center of the support opening 213a and the center of the second opening 212 is arranged/extended along the Z-axis direction.

A size of the support opening 213a is greater than or equal to the outside diameter of the infusion tube 220 and less than the outside diameter of the drip pot 210. Alternately, the bottom of the drip pot 210 may be arranged in the support opening 213a to fix the drip pot 210 210 in the fixing hole 21. When the inner diameter of the fixing hole 21 is greater than the outside diameter of the drip pot 210, the drip pot 210 can be contained and fixed in the fixing hole 21 by placing the bottom of the drip pot 210 in the support opening 213a, so as to prevent the tilt or instability of the drip pot 210 in the fixing hole 21.

Alternately, an inner wall of the support opening 213a may be conical to fit the outer contour of the bottom of the drip pot 210, thus to improve the stability of the drip pot 210 in the fixing hole 21.

Alternately, the support opening 213a may be a cylindrical hole with a diameter of 10 mm (but not limited to that size). The support opening 213a can support the bottom of drip pots 210 of all sizes, so that to fix the drip pots 210 of any size.

The guide channel 213b is connected between the support opening 213a and the first opening 211. The size of the guide channel 213b along the first direction is greater than or equal to the outside diameter of the infusion tube 220 and less than the dimensions of the support opening 213a. The infusion tube 220 enters the fixed hole 21 through the first opening 211 and then enters the support opening 213a through the guide channel 213b.

For example, the guiding channel 213b has a width direction size of 3 mm (but not limited to that size) to facilitate the infusion tube 220 to pass through.

The present application does not specify the extension direction of guide channel 213b. Alternately, the guide channel 213b can be set/extended along the X axis.

Alternately, the guide channel 213b is deviated from the geometric center of the support opening 213a. The center line of the guide channel 213b is not collinear with the geometric center of the support opening 213a, so that the infusion tube 220 contained in the support opening 213a is not easy to move out through the guide channel 213b, and the containment stability of the fixing hole 21 is improved.

Please referring to FIG. 4. the guide channel 213b is deviated to a right circumferential tangent of the support opening 213a to avoid the infusion tube 220 being pulled directly from the guide channel 213b.

In other embodiments, the guide channel 213b may also be extended in a inclined direction, or in a direction of a bend line, or in a direction of a curve, etc., so that the infusion tube 220 located in the supporting opening 213a is not easy to move out through the guide channel 213b, thus improving the containment stability of the fixing hole 21.

Alternately, the size of the first opening 211 along the first direction is greater than the size of the guide channel 213b along the first direction.

Generally, the material of infusion set 200 is mostly made of colorless and transparent chemical materials, and the commonly used interventional catheter irrigation solution is colorless and transparent normal saline. As a result, the drip and liquid level in drip pot 210 cannot be clearly identified, which may lead to excessive dripping speed, excessive cardiac load, and lead to the air enters into blood vessels and resulting in the risk of air embolism.

The infusion set auxiliary device 100 provided in present application can also clearly distinguish the drip and liquid levels in the drip pot 210.

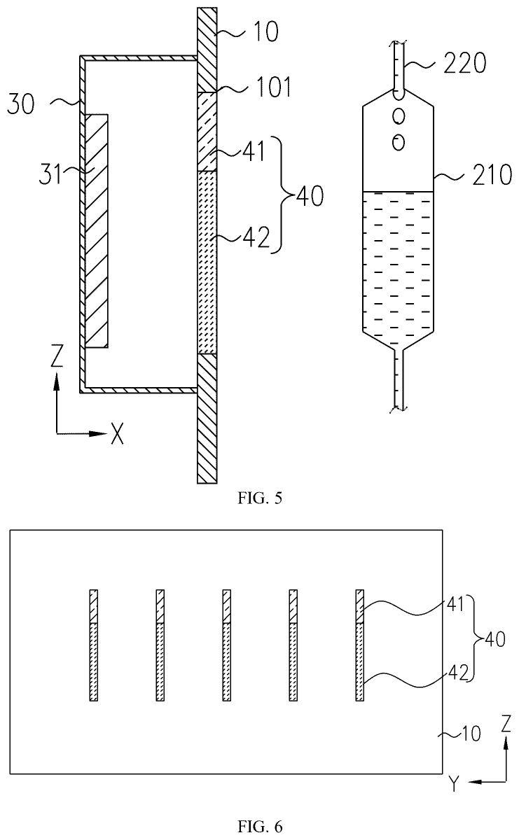

For details, please referring to FIG. 1 and FIG. 5, the bottom plate 10 is provided with a plurality of light-transmitting holes 101 arranged sequentially along the first direction, and each light-transmitting hole 101 is connected with a fixing hole 21. Therein, the light-transmitting hole 101 is in the shape of a long strip and is arranged along the Z-axis direction.

Please referring to FIG. 5, the infusion set auxiliary device 100 further includes a light box 30. The light box 30 is arranged on one side of the bottom plate 10 away from the fixing seat 20. A plurality of light-emitting elements 31 are provided in the light box 30. The light-emitting elements 31 are arranged on a side, of the bottom plate 10, deviating from the fixing seat 20, and plurality of light-emitting elements 31 are corresponded to the plurality of fixing hole 21 one by one, and are corresponded to plurality of light-transmitting holes 101 one by one, and each light-emitting element 31 emits light toward the corresponding fixing hole 21 through the corresponding light-transmitting hole 101.

Alternately, the light-emitting element 31 include but are not limited to LED light beads, LED light strips, etc. Optionally, the light emitted by the light-emitting element 31 is white light.

The present application does not specify the size of light-transmitting holes 101. Alternately, the width of light-transmitting holes 101 along the Y axis is 3.5 mm, and the height of the light-transmitting holes 101 along the Z axis is 50 mm.

Please referring to FIG. 5 and FIG. 6, the infusion set auxiliary device 100 further includes a plurality of light-transmitting glass 40. Each light-transmitting glass 40 is arranged in a light-transmitting hole 101. The light-transmitting glass 40 is embedded in the light-transmitting hole 101.

Please referring to FIG. 5 and FIG. 6, the light-transmitting glass 40 comprises colorless light-transmitting glass 41 and colored light-transmitting glass 42 spliced together. The colored light-transmitting glass 42 is adjacent to the third opening 213 relative to the colorless light-transmitting glass 41. This application does not specify the connection boundary of colorless light-transmitting glass 41 and colored light-transmitting glass 42. Alternately, the area/size of colored translucent glass 42 is greater than that of colorless translucent glass 41. For example, the colorless light-transmitting glass 41 with a height of 15 to 20 mm is installed on the upper part of light transmission hole 101, and the colored light-transmitting glass 42 with a height of 30 to 35 mm is installed on the bottom part of light-transmission hole 101.

The colored light-transmitting glass 42 is set at a position relative to liquid at the bottom (lower) part of the drip pot 210. The colorless light-transmitting glass 41 is set at a position relative to the top (upper) part of the drip pot 210.

The colored light-transmitting glass 42 is a glass that has a certain color and allows the light to pass through, namely transparent or semitransparent. A part of the light emitted by the light-emitting element 31 is transmitted to the liquid of the drip pot 210 through the colored light-transmitting glass 42. The height of the liquid in the drip pot 210 can be clearly observed by using the refraction phenomenon of the liquid, and at this time, the liquid shows the color of the colored light-transmitting glass 42. The other part of the light emitted by the luminescent element 31 is directly transmitted to the drip of the drip pot 210 through the colorless light-transmitting glass 41, so that the drip in the drip pot 210 can be clearly observed.

Further, the colors of the colored light-transmitting glasses 42 in at least two of the light-transmitting holes 101 are different, so that the liquid in at least two drip pot 210 may appear different colors, the colors of the colored light-transmitting glasses 42 may comprise: red, green, blue, purple, orange.

Alternately, the colors of colored light-transmitting glass 42 in different light transmission holes 101are different, that is, each colored light-transmitting glass 42 in the corresponding light transmission hole 101 has a unique color, so that the liquid in different drip pot 210 can be of a different color to facilitate to distinguish different infusion sets 200.

Alternately, the colors of the colored light-transmitting glass 42 in the two adjacent light-transmitting holes 101 are different, so that the liquid in the two adjacent drip pots 210 can appear different colors, so as to facilitate to distinguish two adjacent infusion sets 200.

In addition, number labels can be set at corresponding positions of the fixing holes 21 sequentially to distinguish different drip pots 210 by different number labels, and facilitate the adjustment of the operators or facilitate the operators to communicate with each other and assist the adjustment.

The infusion set auxiliary device 100 provided in present application, can easily and intuitively distinguish drip and liquid levels in the drip pot 210, so as to timely respond to abnormal drip and liquid levels in the drip pot 210 and reduce therapy risks. In addition, it can also make the drip pots 210 of different infusion sets 200 are highly distinguishable, which can be neatly arranged from left to right, and can identify different drip pots 210 by using different number labels, and can also distinguish different drip pot 210 by using different colors, which is convenient for the operators to adjust or communicate with each other and assist the adjustment.

The present application does not specify the material of the bottom plate 10 and the fixing seat 20. Alternately, the material of the bottom plate 10 and the fixing seat 20 include but is not limited to plastic, metal, stainless steel, etc. In this embodiment, the bottom plate 10 and the fixing seat 20 are made of stainless steel matte material, which are prepared by laser cutting technology and are well-made, thus is conducive to long-term use.

The drip rate of a general infusion set 200 usually is manually controlled, and the amount of infusion per minute is estimated according to experience, which is not standardized, resulting in inaccurate drip rate; In addition, when the amount of suspension fluid, namely the fluid in the infusion bottle, reaches 500 ml, it is sometimes necessary to pressurize, and when the pressure cannot be stopped in time after the liquid is transferred, the risk of air embolism may occur.

In one possible embodiment, the infusion set auxiliary device 100 further includes a drip controller (not shown), which is located outside the infusion tube 220 at the top of the drip pot 210 to electronically control the drip rate of the drip in the drip pot 210 instead of a traditional manual control device for the drip rate of the infusion set.

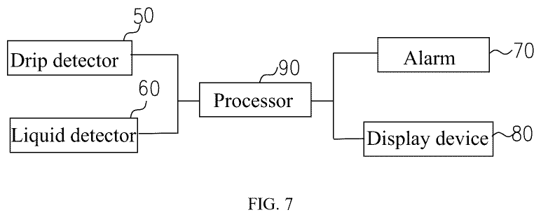

Referring to FIG. 7, the infusion set auxiliary device 100 for vascular intervention provided in present application also includes at least one drip detector 50. Each drip detector 50 is arranged on the inner wall of the corresponding fixing hole 21. The drip detector 50 is set relative to the drip of the drip pot 210 to detect the drip rate of the drip in the drip pot 210. Alternately, the number of the at least one drip detector 50 is equal to the number of the fixing hole 21, and each drip detector 50 corresponds with one fixing hole 21.

Alternately, the drip detector 50 includes, but is not limited to, photoelectric non-contact sensors, such as infrared sensors, which include a transmitter and a receiver, respectively located on opposite sides of the inner wall of the fixed hole 21, and the transmitter and receiver are respectively located on opposite sides of drip pot 210 and near the top of the drip pot 210. The detection principle of infrared sensor includes but is not limited to: the light emitted by the transmitter passes through a drop path of the drip, and the light emitted by the transmitter is received by the receiver when the light emitted by the emitter does not pass through the drip. When the light emitted by the emitter passes through the drip, the receiver does not receive the light from the emitter, and the infrared sensor detects the dynamics of the drip, the drip rate per minute, and the drip rate is more accurate and standardized than the results determined by humans.

The infrared sensor can be powered by silicon photovoltaic cells or other batteries.

Please referring to FIG. 7, the infusion set auxiliary device 100 further includes at least one liquid detector 60. Each liquid detector 60 can be arranged on the inner wall of the fixing hole 21 or outside the fixing hole 21. When the infusion tube 220 at the top of the drip pot 210 is contained in fixed hole 21, the liquid detector 60 may be arranged on the inner wall of fixing hole 21 and arranged on opposite sides of the infusion tube 220 at the top of the drip pot 210, or the liquid detector 60 may be clamped on opposite sides of the infusion tube 220 at the top of the drip pot 210. When the infusion tube 220 at the top of the drip pot 210 is positioned outside the fixing hole 21, the liquid detector 60 is clamped on opposite sides of the infusion tube 220 at the top of the drip pot 210. Alternately, the number of the at least one liquid detector 60 is equal to the number of the fixing hole 21, and each liquid detector 60 corresponds with one fixing hole 21.

The liquid detector 60 is arranged relative to the infusion tube 220 connected to the top of the drip pot 210. The liquid detector 60 is used to detect whether the infusion tube 220 at the top of the drip pot 210 has a liquid. The liquid detector 60 includes, but is not limited to, photoelectric non-contact sensors, such as infrared sensors, whose detection principle may be the same or different from that of the drip detector 50.

Alternately, please referring to FIG. 7, the infusion set auxiliary device 100 further includes an alarm 70. The alarm 70 is electrically connected to the drip detector 50 and the liquid detector 60. The alarm 70 is used to generate an alarm signal when the drip rate detected by the drip detector 50 is 0 and the liquid in the infusion tube 220 detected by the liquid detector 60 is empty. Alternately, the alarm 70 is electrically connected to all of the drip detector 50 and all of the liquid detector 60, and the alarm 70 generates the alarm signal when the drip rate detected by the drip detector 50 corresponding to one fixing hole 21 is 0 and the liquid in the infusion tube 220 detected by the liquid detector 60 corresponding to the same fixing hole 21 is empty.

Alternately, the alarm 70 includes, but is not limited to, an audible alarm or a warning light. When the drip rate detected by the drip detector 50 is 0 and the liquid in the infusion tube 220 detected by the liquid detector 60 is empty, the alarm 70 generates a light or audible warning so that the operator can observe that the drip rate detected by the drip detector 50 is 0 and the liquid in the infusion tube 220 detected by the liquid detector 60 is empty. Therefore, corresponding operations can be executed by the operator in time to avoid air embolism.

Alternately, please referring to FIG. 7, the infusion set auxiliary device 100 further includes a display device 80. The display device 80 is electrically connected to the drip detector 50 and the liquid detector 60. The display device 80 is used to display the drip rate in the drip pot 210 and information about whether there is liquid in the infusion tube 220 connected to the top of the drip pot.

The infusion set auxiliary device 100 further comprises a processor 90, the display device 80, the drip detector 50 and the liquid detector 60 are all electrically connected to the processor 90. The drip detector 50 and the liquid detector 60 are used for sending corresponding detection result to the processor 90, the processor 90 processes the test results and controls the display device 80 to display the drip rate in the drip pot 210 and information about whether there is liquid in the infusion tube 220 connected to the top of the drip pot, after processing the test results, so that the user or the operator can observe such information directly. Alternately, the processor 90 is electrically connected to all of the drip detector 50 and all of the liquid detector 60, and the processor 90 display the drip rate in the drip pot 210 contained in one fixing opening 21 and the information about whether there is liquid in the infusion tube 220 connected to the top of the drip pot 210 contained in the same fixing opening 21 together.

The number of the display device 80 can be one or more, for example, the number of the display device 80 can be two, one is located in the ward, the other is located in the work station of the medical staff, so that the medical staff can monitor the infusion situation in real time.

The infusion set auxiliary device 100 provided in present application may include either or both of the alarm 70 and the display device 80.

Please referring back to FIG. 2. the present application also provides an infusion set system 1000 for vascular intervention, the infusion set system 1000 includes a plurality of infusion sets 200 and the infusion set auxiliary equipment 100 described in any of the above embodiments. The infusion set 200 comprises a drip pot 210, which is arranged in the fixing hole 21 of the infusion set auxiliary device 100, and the infusion set auxiliary device 100 can arrange a plurality of drip pot 210 of the infusion set 200 neatly, and facilitate to organize multiple infusion sets 200, make it easy to distinguish the drip and liquid level in the drip pots 210 of multiple infusion sets 200, and accurately obtain the situation that the drip rate of the drip pot 210 is 0 and the liquid of the infusion tube 220 at the top of the drip pot 210 is empty, and can send an alarm signal to remind medical staff to respond in time and reduce the risk of air embolism.

The above descriptions are only optional implementations of the disclosure, and are not intended to limit the present disclosure. Any modifications, equivalent replacements, improvements, etc. made within the spirit and principles of the present disclosure shall be included in the protection range of the present disclosure.

What is claimed is:

1. An infusion set auxiliary device for vascular intervention, wherein the infusion set auxiliary device comprises:
a bottom plate;
a fixing seat, arranged on the bottom plate, the fixing seat has a top surface, a bottom surface opposite to the top surface, and a side surface connected between the top surface and the bottom surface, the side surface is arranged opposite to the bottom plate, the fixing seat is provided with a plurality of fixing holes arranged in a first direction sequentially, each fixing hole is at least used to contain a drip pot of an infusion set;
wherein each fixing hole penetrates through the side surface and forms a first opening on the side surface, and a size of the first opening along the first direction is greater than or equal to the outside diameter of an infusion tube of the infusion set and less than the outside diameter of the drip pot;
wherein each fixing hole penetrates through the top surface and forms a second opening on the top surface, the second opening is connected with the first opening, and a size of the second opening is greater than or equal to the outside diameter of the drip pot;
wherein the fixing hole penetrates through the bottom surface and forms a third opening on the bottom surface, the third opening is connected with the first opening, and a size of the third opening is greater than or equal to the outside diameter of the infusion tube and less than the outside diameter of the drip pot.

2. The infusion set auxiliary device of claim 1, wherein the third opening comprises a support opening and a guide channel, the support opening is concentrically arranged with the second opening, the guide channel is connected between the support opening and the first opening, and a size of the support opening is greater than or equal to the outside diameter of the infusion tube and less than the outside diameter of the drip pot, a size of the guide channel along the first direction is greater than or equal to the outside diameter of the infusion tube and less than the size of the support opening.

3. The infusion set auxiliary device of claim 2, wherein the guide channel is deviated from the geometric center of the support opening.

4. The infusion set auxiliary device of claim 2, wherein the guide channel is extended in a inclined direction, or in a direction of a bend line, or in a direction of a curve.

5. The infusion set auxiliary device of claim 2, wherein the size of the first opening along the first direction is greater than the size of the guide channel along the first direction.

6. The infusion set auxiliary device of claim 1, wherein the bottom plate is provided with a plurality of light-transmitting holes arranged sequentially along the first direction, each light-transmitting hole is connected with one corresponding fixing hole;
wherein the infusion set auxiliary device further comprises a plurality of light-emitting elements arranged on a side of the bottom plate deviating from the fixing seat, and each light-emitting element emits light toward to the corresponding fixing hole through the corresponding light-transmitting hole.

7. The infusion set auxiliary device of claim 6, wherein the infusion set auxiliary device further comprises a plurality of light-transmitting glasses, each light-transmitting glass is arranged in one corresponding light-transmitting hole.

8. The infusion set auxiliary device of claim 7, wherein the light-transmitting glass comprises a colorless light-transmitting glass and a colored light-transmitting glass spliced together, the colored light-transmitting glass is adjacent to the third opening relative to the colorless light-transmitting glass, and the colored light-transmitting glass sets at a position at least relative to liquid at a bottom of the drip pot.

9. The infusion set auxiliary device of claim 8, wherein colors of the colored light-transmitting glasses in at least two of the light-transmitting holes are different.

10. The infusion set auxiliary device of claim 8, wherein colors of the colored light-transmitting glasses in different light transmission holes are different.

11. The infusion set auxiliary device of claim 8, wherein colors of the colored light-transmitting glasses in two adjacent light-transmitting holes are different.

12. The infusion set auxiliary device of claim 1, wherein the infusion set auxiliary device further comprises at least one drip detector each arranges on an inner wall of the corresponding fixing hole, and the drip detector is set relative to the drip of the corresponding drip pot to detect the drip rate of the drip in the drip pot.

13. The infusion set auxiliary device of claim 12, wherein the infusion set auxiliary device further comprises at least one liquid detector each sets with respect to an infusion tube connected to a top of the corresponding drip pot, and the liquid detector is used for detecting whether the infusion tube at the top of the drip pot has liquid.

14. The infusion set auxiliary device of claim 13, wherein the infusion set auxiliary device further comprises an alarm, the alarm is electrically connected with the drip detector and the liquid detector and is used to generate an alarm signal when the drip rate detected by the drip detector is 0 and the liquid in the infusion tube detected by the liquid detector is empty.

15. The infusion set auxiliary device of claim 13, wherein the infusion set auxiliary device further comprises a display device, the display device is electrically connected to the drip detector and the liquid detector, and the display device is used to display the drip rate in the drip pot and display information about whether there is liquid in the infusion tube connected to the top of the drip pot.

16. An infusion set system for vascular intervention, wherein the infusion set system comprises a plurality of infusion sets and an infusion set auxiliary device, each infusion set comprises a drip pot; wherein the infusion set auxiliary device comprises:
a bottom plate;
a fixing seat, arranged on the bottom plate, the fixing seat has a top surface, a bottom surface opposite to the top surface, and a side surface connected between the top surface and the bottom surface, the side surface is arranged opposite to the bottom plate, the fixing seat is provided with a plurality of fixing holes arranged in a first direction sequentially, each fixing hole is at least used to contain the drip pot of one infusion set;
wherein each fixing hole penetrates through the side surface and forms a first opening on the side surface, and a size of the first opening along the first direction is greater than or equal to the outside diameter of an infusion tube of the infusion set and less than the outside diameter of the drip pot;
wherein each fixing hole penetrates through the top surface and forms a second opening on the top surface, the second opening is connected with the first opening, and a size of the second opening is greater than or equal to the outside diameter of the drip pot;
wherein the fixing hole penetrates through the bottom surface and forms a third opening on the bottom surface, the third opening is connected with the first opening, and a size of the third opening is greater than or equal to the outside diameter of the infusion tube and less than the outside diameter of the drip pot.

17. The infusion set system of claim 16, wherein the third opening comprises a support opening and a guide channel, the support opening is concentrically arranged with the second opening, the guide channel is connected between the support opening and the first opening, and a size of the support opening is greater than or equal to the outside diameter of the infusion tube and less than the outside diameter of the drip pot, a size of the guide channel along the first direction is greater than or equal to the outside diameter of the infusion tube and less than the size of the support opening.

18. The infusion set system of claim 17, wherein the guide channel is deviated from the geometric center of the support opening.

19. The infusion set system of claim 17, wherein the guide channel is extended in a inclined direction, or in a direction of a bend line, or in a direction of a curve.

20. The infusion set system of claim 17, wherein the size of the first opening along the first direction is greater than the size of the guide channel along the first direction.
